(19) United States
(12) Patent Application Publication
(10) Pub. No.: US 2024/0342369 A1
(43) Pub. Date: Oct. 17, 2024

(54) ROLLER CLAMP CONTROL MECHANISM

(71) Applicant: CareFusion 303, Inc., San Diego, CA (US)

(72) Inventors: Jason Andrew WINE, Brea, CA (US); Michael B. GOGARTY, Oceanside, CA (US)

(21) Appl. No.: 18/632,755

(22) Filed: Apr. 11, 2024

Related U.S. Application Data
(60) Provisional application No. 63/459,508, filed on Apr. 14, 2023.

(51) Int. Cl. A61M 5/168; A61M 39/22; A61M 39/28

(52) U.S. Cl. CPC A61M 5/16813; A61M 39/286; A61M 2039/226; A61M 2205/103

(57) ABSTRACT

A roller clamp control mechanism which adapts to the roller clamp to control the position of the roller clamp and adjust the fluid flow rate in a connector tube of an infusion set is provided. The mechanism may include a roller clamp, a roller wheel, a control unit, rotating bearings, a body fixture, and a linear drive to control the position of the roller wheel on the connector tube. Different assemblies for the roller clamp control mechanism are shown and described, all of which allow for changing the clamping pressure on the connector tube using a control unit. Infusion sets and methods of adjusting fluid flow rates are also provided.

### CROSS REFERENCE TO RELATED APPLICATION

This application claims the benefit of priority under 35 U.S.C. § 119 to U.S. Provisional Patent Application Ser. No. 63/459,508, entitled “ROLLER CLAMP CONTROL MECHANISM”, filed on Apr. 14, 2023, the entire contents of which is incorporated herein by reference.

### TECHNICAL FIELD

The present disclosure generally relates to a gravity intravenous (IV) set or infusion pump flow control device, and in particular a precision roller clamp control mechanism.

### BACKGROUND

Flow controllers in the form of roller clamps are used in the medical field for intravenous (IV) applications. Typical roller clamps control a flow rate through an IV tube by clamping the tube in between a roller wheel and a housing. This approach, for one, provides a limited range of flow rate control because the roller wheel is essentially too sensitive in that a small movement of the roller wheel or dimension change causes a large change in flow rate of the fluid through the tube. Thus, the relatively course flow rate change provided by a typical roller clamp makes it difficult to provide precise flow control.

Also, typical roller clamps have flow rate drifting issues based on slippage of the roller wheel, such as when fluid pressure in the tube causes the roller wheel to roll back from the adjusted position. Further, typical roller clamps are sized for a specific IV set tube dimension, which requires having different sized roller clamps for use with various IV set tube dimensions.

Thus, it is desirable to provide a precision roller wheel assembly that works with multiple IV tube sizes, provides quick course flow rate adjustments and fine flow rate adjustments, and eliminates or minimizes roller wheel slippage, that also can be machine controlled by a control unit in a gravity IV set.

### SUMMARY

One or more embodiments provide a roller clamp control mechanism assembly. The roller clamp assembly includes a housing configured to receive a portion of a connector tube of an infusion set. The housing may comprising two opposing side walls spaced apart from each other, each side wall having an opposing guide groove longitudinally positioned in an interior surface, a guide wall disposed between the side walls, the guide wall converging along its length toward the position of the guide grooves, a roller wheel having two axial projections slidingly seated in the guide grooves, the roller configured to move along a longitudinal axis of the housing as the projections slide in the guide grooves, wherein spacing between the guide wall and the roller wheel decreases over a length of the guide wall, and a roller clamp control mechanism which adapts to the roller clamp to control the position of the roller clamp.

In some embodiments, the roller clamp control mechanism comprises a control unit, two rotating bearings fixed to a housing of the control unit, and a body fixture attached to the roller clamp and positioned into the control unit which is connected to a linear drive, wherein the roller clamp is placed into the housing of the control unit such that the roller wheel is between the two rotating bearings, and wherein as the linear drive actuates the body fixture is driven forwards or backwards, while the roller wheel stays stationary with respect to the control unit, thereby changing the clamping pressure on the connector tube.

In some embodiments, the body fixture attaches to the roller clamp via a mechanical interlock or surface friction. In some embodiments, sliding of the roller wheel in a direction of lesser spacing between the guide wall and the roller wheel causes the roller wheel to impinge on the connector tube to a gradually increasing extent, wherein the increased impingement of the roller wheel on the connector tube is configured to reduce a fluid flow rate through the connector tube. In some embodiments, the rotating bearings have a gear pattern on the surface that interlocks with a gear pattern on the surface of the roller wheel. In some embodiments, the roller wheel is configured as a substantially binary flow switch, for example, where the flow rate is either wide open, adjusted down to a specific flow rate, or a fully blocked flow rate. In some embodiments, a spring pin that connects the control unit to the linear drive.

In some embodiments, the roller clamp control mechanism comprises a control unit, and two rotating bearings fixed within a bearing housing, wherein the bearing hosing is connected to a linear drive, and wherein as the linear drive actuates, the bearing housing is moved back and forth as desired which adjusts the roller wheel's position on the connector tube. In some embodiments, the rotating bearings attach to the roller clamp via a mechanical interlock or surface friction. In some embodiments, sliding of the roller wheel in a direction of lesser spacing between the guide wall and the roller wheel causes the roller wheel to impinge on the connector tube to a gradually increasing extent, wherein the increased impingement of the roller wheel on the connector tube is configured to reduce a fluid flow rate through the connector tube. In some embodiments, the roller clamp assembly further comprises a spring pin that attaches the roller clamp to the control unit.

In some embodiments, the roller clamp control mechanism comprises two rotating bearings each fixed within a respective bearing arm which pivots about a fixed axis of the bearing arm, and a bearing clearance path for the rotating bearings to slide through, wherein bearing clearance path allows the bearing arm of each rotating bearing to rotate inward but not in outward, and wherein the roller clamp assembly is able to slide over the roller wheel and adjust the roller wheel position. In some embodiments, sliding of the roller wheel in a direction of lesser spacing between the guide wall and the roller wheel causes the roller wheel to impinge on the connector tube to a gradually increasing extent, wherein the increased impingement of the roller wheel on the connector tube is configured to reduce a fluid flow rate through the connector tube. In some embodiments, a means of the roller wheel connection of the roller clamp assembly is agnostic to an initial position of the roller wheel. In some embodiments, the roller clamp assembly further comprising a bearing arm return spring configured to bias the bearing arms to return to an upright position.

Embodiments of the present disclosure provide a gravity infusion set comprising a piercing spike, a drop chamber, a connector tube, a fitting, and a roller clamp assembly, the roller clamp assembly comprising a housing configured to receive a portion of a connector tube of an infusion set, the housing comprising two opposing side walls spaced apart from each other, each side wall having an opposing guide groove longitudinally positioned in an interior surface, a guide wall disposed between the side walls, the guide wall converging along its length toward the position of the guide grooves, a roller wheel having two axial projections slidingly seated in the guide grooves, the roller configured to move along a longitudinal axis of the housing as the projections slide in the guide grooves, wherein spacing between the guide wall and the roller wheel decreases over a length of the guide wall, and a roller clamp control mechanism which adapts to the roller clamp to control the position of the roller clamp.

Embodiments of the present disclosure provide a method of adjusting a fluid flow rate through a connector tube coupled to a fluid source, the method comprising inserting the connector tube through a housing of a roller clamp assembly having a roller wheel and a roller clamp control mechanism which adapts to the roller clamp to control the position of the roller clamp, controlling the roller clamp control mechanism using a control unit to compress the connector tubing with the roller wheel to produce an adjustment that causes a change in the fluid flow rate through the connector tube.

In some embodiments, the roller clamp control mechanism comprises a control unit, two rotating bearings fixed to a housing of the control unit, and a body fixture attached to the roller clamp and positioned into the control unit which is connected to a linear drive, wherein the roller clamp is placed into the housing of the control unit such that the roller wheel is between the two rotating bearings, and wherein as the linear drive actuates the body fixture is driven forwards or backwards, while the roller wheel stays stationary with respect to the control unit, thereby changing the clamping pressure on the connector tube.

In some embodiments, the roller clamp control mechanism comprises a control unit and two rotating bearings fixed within a bearing housing, wherein the bearing hosing is connected to a linear drive, and wherein as the linear drive actuates, the bearing housing is moved back and forth as desired which adjusts the roller wheel's position on the connector tube. In some embodiments, the roller clamp control mechanism comprises two rotating bearings each fixed within a respective bearing arm which pivots about a fixed axis of the bearing arm, and a bearing clearance path for the rotating bearings to slide through, wherein bearing clearance path allows the bearing arm of each rotating bearing to rotate inward but not in outward, and wherein the roller clamp assembly is able to slide over the roller wheel and adjust the roller wheel position.

The foregoing and other features, aspects and advantages of the disclosed embodiments will become more apparent from the following detailed description and accompanying drawings.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure.

FIG. 1A depicts a perspective view of an example infusion set having a typical roller clamp.

FIG. 1B depicts a perspective view of an example infusion set having a roller clamp control mechanism in accordance with aspects of the present disclosure.

FIG. 2 depicts a perspective view of an example roller clamp in accordance with aspects of the present disclosure.

FIG. 3 depicts a side view of a precision roller clamp control mechanism.

FIG. 4 depicts a side view of a precision roller clamp control mechanism.

FIG. 5 depicts a side view of a precision roller clamp control mechanism.

FIG. 6 depicts a perspective view of a precision roller clamp control mechanism of FIG. 5.

FIG. 7 depicts a front view of a precision roller clamp control mechanism of FIG. 5.

FIG. 8 depicts a perspective view of a precision roller clamp control mechanism of FIG. 5.

FIG. 9 depicts a side view of a precision roller clamp control mechanism of FIG. 5.

FIG. 10 depicts a side view of a precision roller clamp control mechanism of FIG. 5.

FIG. 11 depicts a side view of a precision roller clamp control mechanism of FIG. 5.

FIG. 12 depicts a side view of a precision roller clamp control mechanism of FIG. 5.

FIG. 13 depicts a side view of a precision roller clamp control mechanism of FIG. 5.

FIG. 14 depicts a side view of a precision roller clamp control mechanism of FIG. 5.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

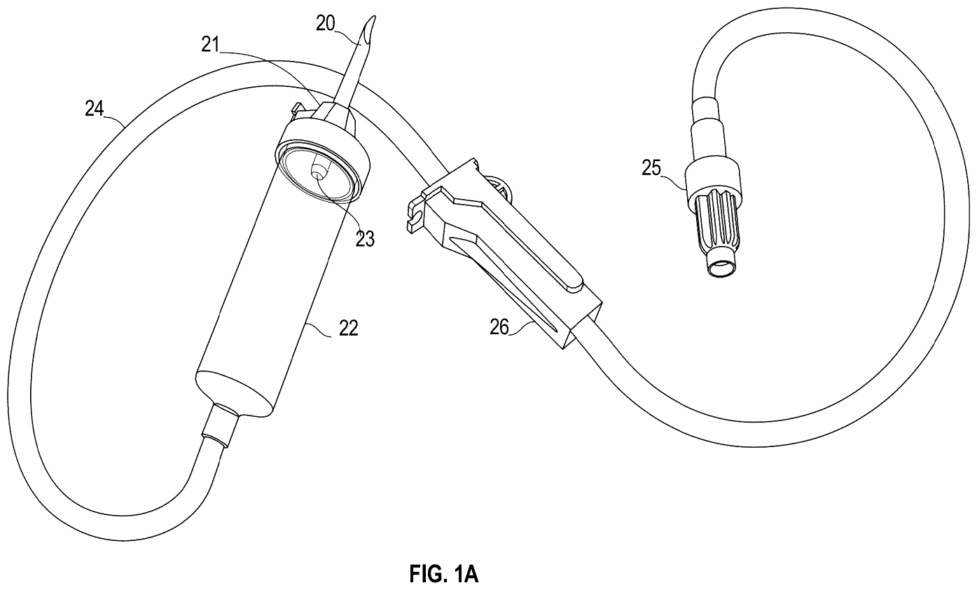

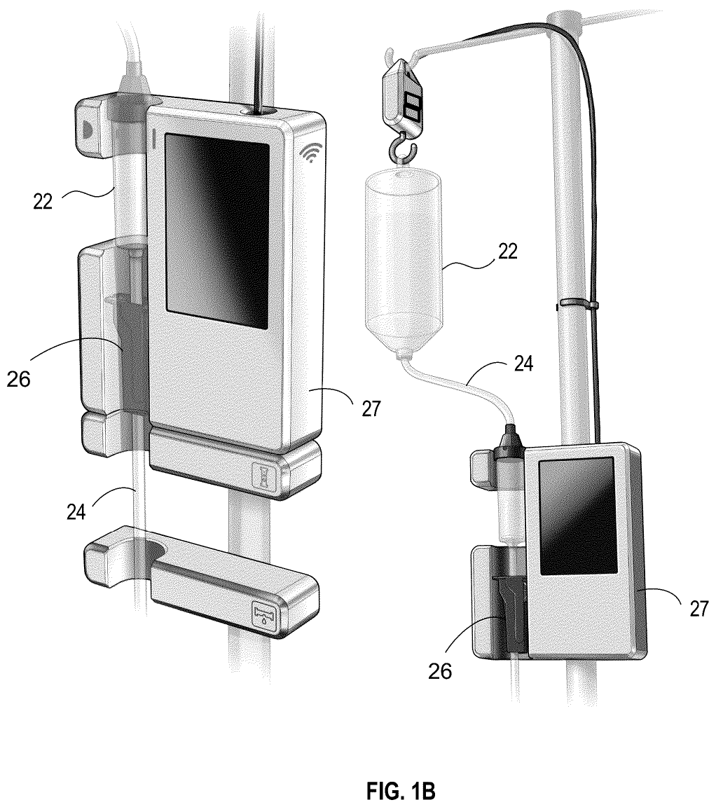

The present disclosure relates to a roller clamp and in particular to a roller clamp control mechanism that can be machine controlled for use with a roller clamp in a gravity IV set. The roller clamp regulates the flow rate of a medical fluid (for example a solution of a drug to be administered to a patient, or blood) flowing through a tube. A standard infusion set may be used to infuse the fluid. An example of a standard infusion set is shown in FIG. 1A.

The infusion set may include a piercing spike 20 which may either be a sharp spike for piercing rubber stoppers or rounded and blunt for insertion into a bag. The spike may contain one channel for fluid and optionally a second channel for venting. A vent 21 may be present in the vicinity of the piercing spike to allow air to flow into the drop chamber 22. The vent 21 may be provided with a bacterial filter to prevent bacteria from entering the equipment.

The drop chamber 22 has a drop generator 23 at the top of the drop chamber 22 that produces drops of a certain size. Drops from the drop generator 23 fall into the drop chamber 22 such that the drop chamber 22 is partially filled with liquid. This prevents air bubbles from entering the connector tube 24, which would be harmful to a patient. A particle filter may be provided at the lower aperture of the drop chamber 22.

In some embodiments, the connector tube 24 connects the drop chamber 22 with the patient. The connector tube 24 may be around 150 cm long and can be manufactured from PVC. The tube 24 is shown shortened in FIG. 1A for clarity. The connector tube 24 typically has a continuous diameter throughout the length of the tube.

At the end of the connector tube 24 is a Luer fitting 25 which is standardized for connection to all other pieces of apparatus having a standard Luer cone. The person skilled in the art will appreciate that the Luer fitting 25 can be fitted to a hypodermic needle (not shown) for infusing the medical fluid into the circulatory system of a patient (e.g., into a vein). Between the drop chamber 22 and the Luer fitting 25 and engaging with the connector tube 24, is a roller clamp 26.

Roller clamp mechanisms in accordance with embodiments of the present disclosure may be used with the gravity IV infusion set shown in FIG. 1B. In some embodiments, the standard infusion set includes drop chamber 22, roller clamp assembly 26, connector tube 24, as described above, and a control unit 27. The control unit 27 may control the roller clamp in accordance with aspects of the present disclosure.

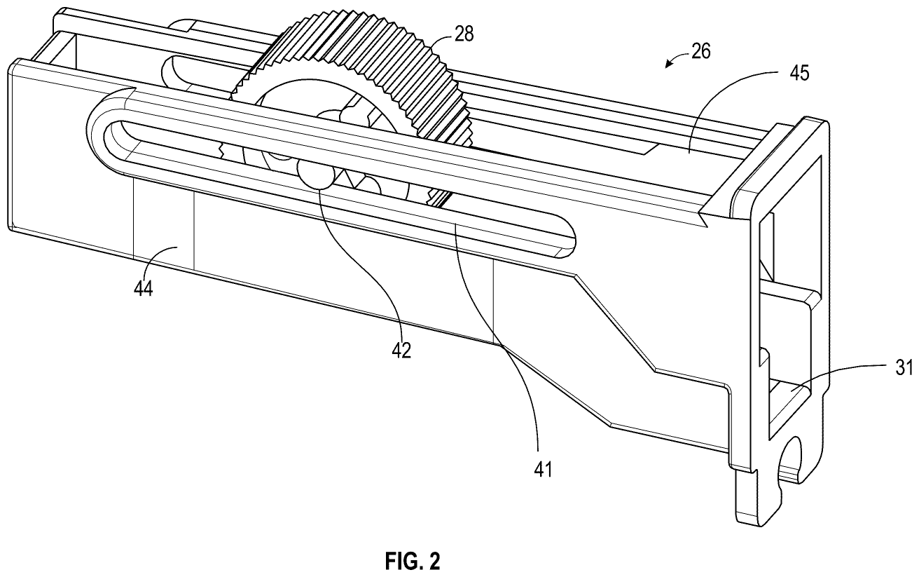

A non-limiting example of a roller clamp assembly 26 is illustrated in FIG. 2 and may be used with roller clamp control mechanisms described herein. Roller clamp assembly 26 may have two opposing side walls 44 and 45 having a pair of guide grooves 41 that are aligned with each other and face each other. A flow-regulating roller 28 may be provided having axially-projecting shafts 42 protruding from the centers of each side of the roller 28. The shafts 42 of the roller 28 may be captured by and seated in the guide grooves 41 so that the roller 28 can move up and down the guide grooves 41.

In some embodiments, the entire roller clamp assembly 26 has four walls (see FIG. 2) in an open-ended boxlike construction and is dimensioned and configured to receive a connector tube. In use, the tube 24 passes through the roller clamp assembly 26, between the two opposing side walls 44 and 45, the roller 28 and a guide wall 31 that is opposed to the roller 28.

In some embodiments, in the roller clamp assembly 26, the surface of the guide wall 31 may converge along its length toward the position of the guide grooves 41 in the direction of the guide grooves 41. This tends to urge the connector tube 24 within the roller clamp assembly 26 toward the guide grooves 41 and thus toward roller 28.

Thus, rolling the roller 28 downwardly along the guide grooves 41 in the direction of the gradually closer guide wall 31 in the direction of the arrows causes the roller 28 to impinge against the connector tube 24. As the roller 28 impinges on the tube 24, the tube 24 becomes squeezed, as it is a flexible material such as PVC, and the lumen of the infusion tube 24 therefore becomes smaller. In this way, by narrowing of the lumen, the flow rate of liquid passing through the connector tube 24 can be regulated.

Thus, the roller clamp assembly 26 controls the flow rate through the infusion tube 24 by clamping the infusion tube 24 between the roller 28 and the guide wall 31. As discussed above, this provides for a flow rate change because a small movement of the roller 28 causes a large change in the flow rate of the fluid through the tube 24. For example, with the roller 28 in a wide-open position where the roller 28 does not impinge on the tube 24, a fluid flow rate may be anywhere from 2,000 to 8,000 milliliters per hour (ml/hr). This flow rate may be too fast to count drips in a drip chamber (e.g., drop chamber 22) as the maximum flow rate for counting drops may be 250 ml/hr.

Also, the force of the fluid in the tube 24 exerts a biasing force against the roller 28, which often leads to slippage of the roller 28 (e.g., the roller 28 rolls back) from the adjusted position. In addition, the roller 28 needs to be sized for the tube 24 dimensions, so if a different size (e.g., diameter) tubing is used, a different size roller must then be used as well.

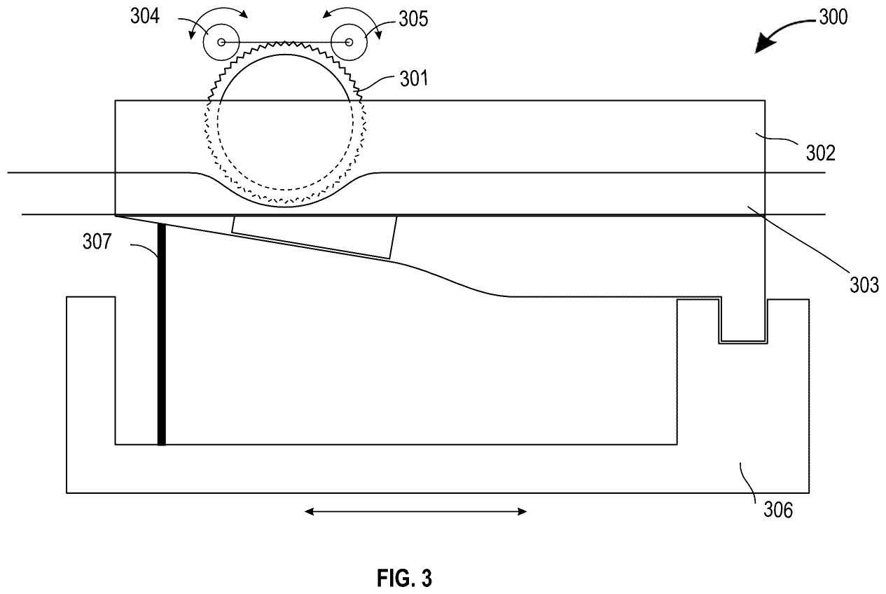

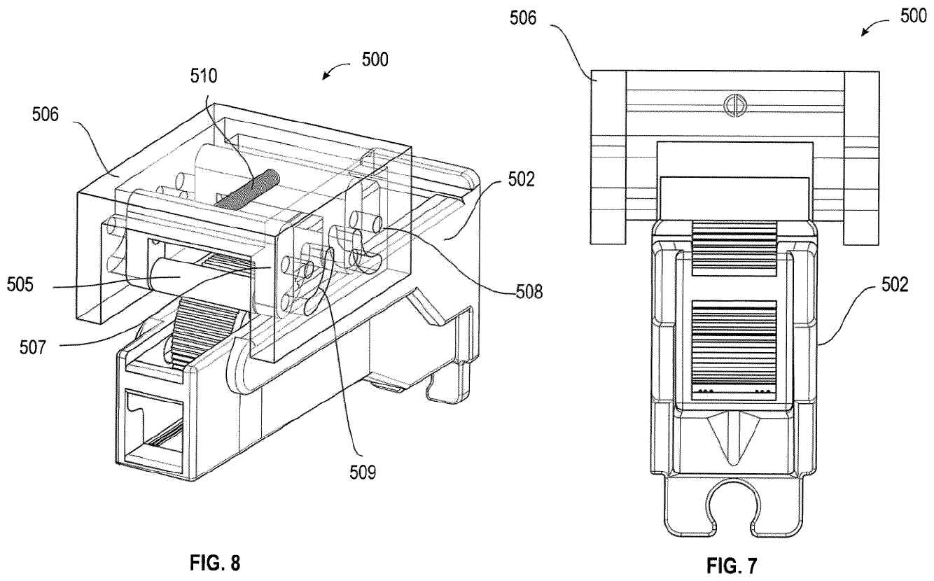

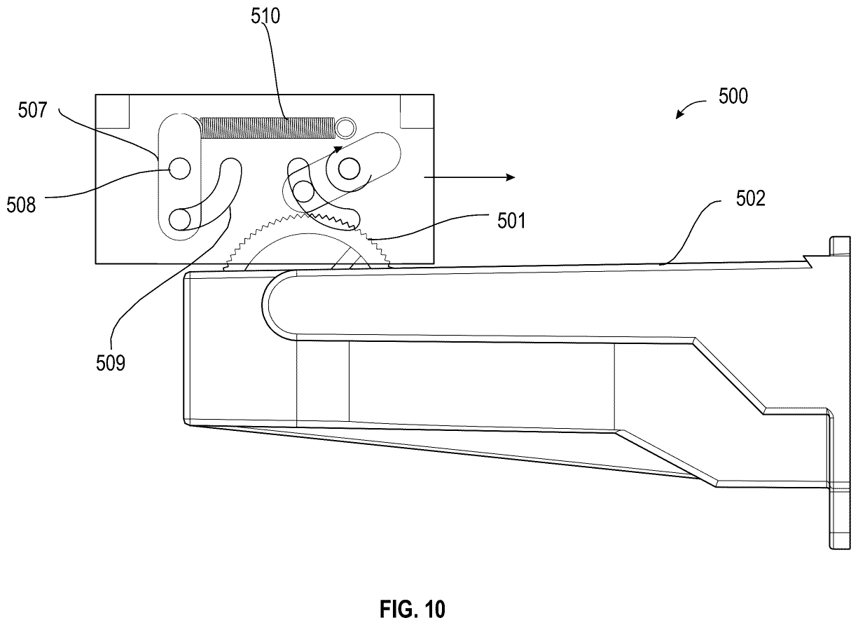

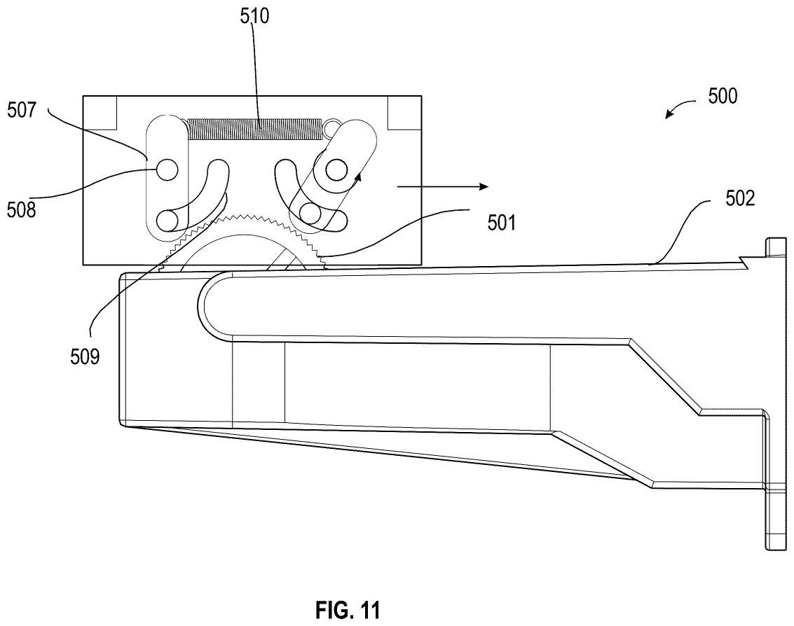

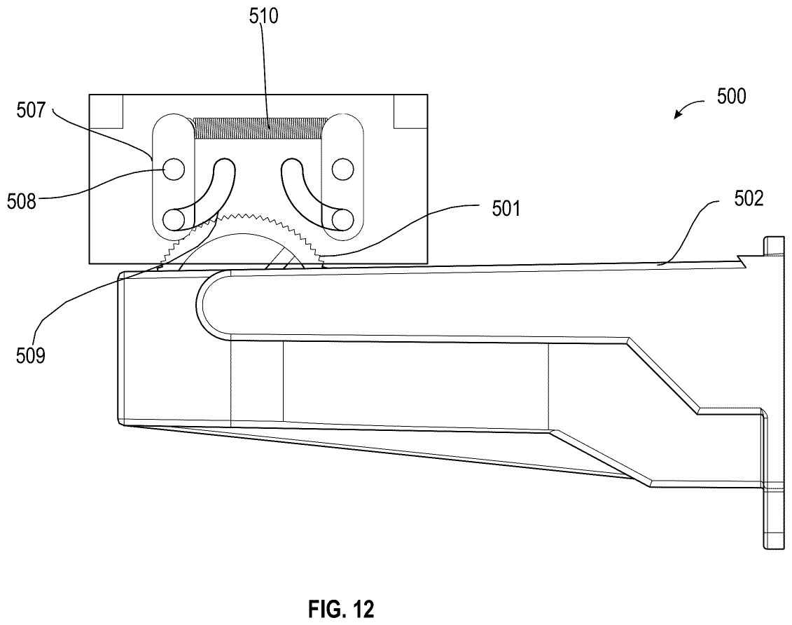

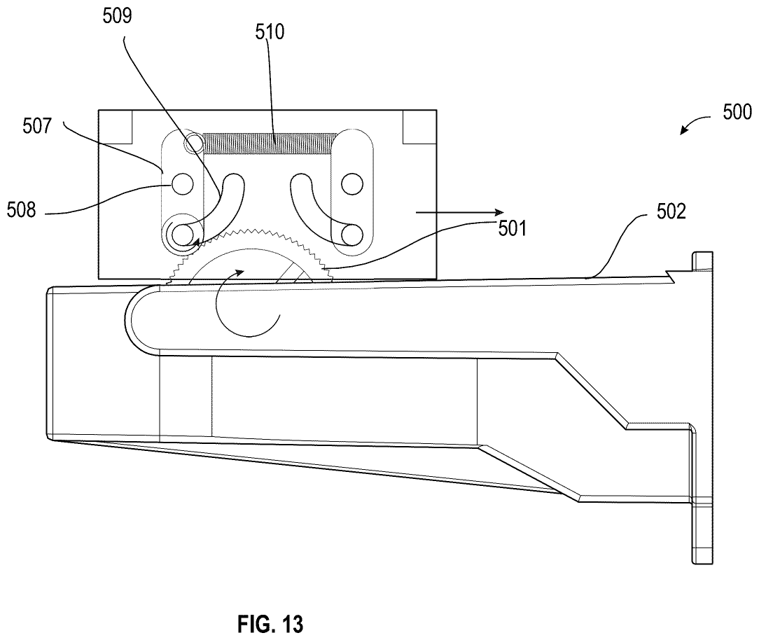

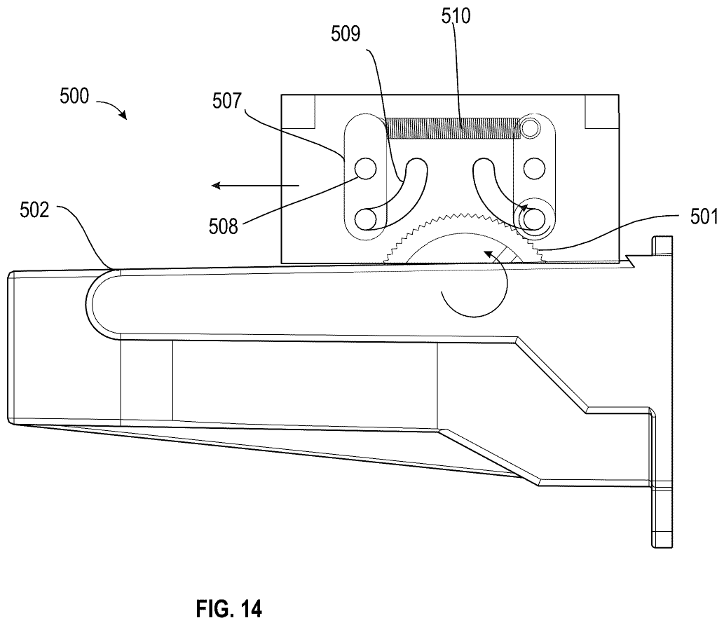

With reference to FIGS. 3-14, examples of roller clamp assembly control mechanisms that allow for the precise control of a roller clamp assembly 26 by control unit 27 are shown.

The roller clamp control mechanism assembly 300 illustrated in FIG. 3 may have a body fixture 302 comprising two opposing side walls and a roller clamp assembly 26 may be seated inside the body fixture. In some embodiments, the roller clamp assembly 26 comprises roller wheel 301. In some embodiments, two rotating bearings 304 and 305 may be fixed to a housing of the control unit (control unit 27 not shown in FIG. 3 for simplicity). In some embodiments, rotating bearings 304 and 305 have a smooth surface. In some embodiments, rotating bearings 304 and 305 may have a gear pattern on the surface that interlocks with a gear pattern on roller wheel 301. In some embodiments, the body fixture 302 is attached to the roller clamp and positioned into the control unit 27 which is connected to a linear drive 306 by spring pin 307. The roller clamp assembly 26 may be placed into the control unit 27 housing such that the roller wheel 301 is between the two rotating bearings 304 and 305. In some embodiments, as the linear drive 306 actuates, the body fixture 302 is driven forwards or backwards, while the roller wheel 301 stays stationary with respect to the control unit 27, thereby changing the clamping pressure on the connector tube 303. This change in clamping pressure closes or opens the flow, respectively. Accordingly, the roller clamp control mechanism assembly 300, controlled by control unit 27, effectively limits the flow through the connector tubing 303 to the desired rate.

In some embodiments, the body fixture 302 attaches to the roller clamp assembly 26 via a mechanical interlock, or surface friction (e.g., rubber pads under sufficient spring preload to firmly grasp the roller body housing and not slip).

The fluid flow rate may be quickly adjusted by way of the control unit 27 to a fully blocked flow rate of 0 ml/hr (e.g., quick occlusion), or any other desired fluid flow rate between 250 ml/hr and 0 ml/hr (e.g., 50 ml/hr, 125 ml/hr), when the roller wheel 301 is moved into the fully engaged position. Thus, the roller wheel 301 may be configured as a substantially binary flow switch (e.g., on/off switch), for example, where the flow rate is either wide open or adjusted down to a specific flow rate such as 250 ml/hr, or even to a fully blocked flow rate of 0 ml/hr.

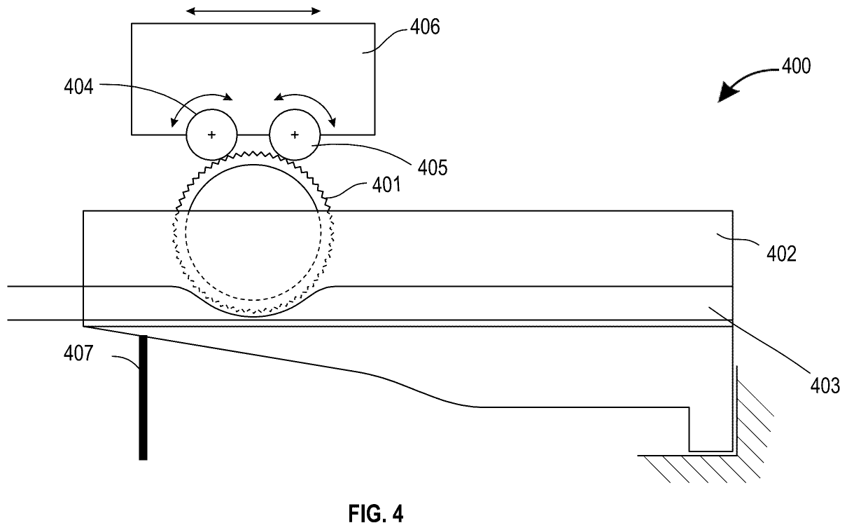

The roller clamp control mechanism assembly 400 illustrated in FIG. 4 may have a body fixture 402 comprising two opposing side walls and a roller clamp assembly 26 that is seated inside the body fixture 402. In some embodiments, the roller clamp assembly 26 comprises roller wheel 401. In some embodiments, two rotating bearings 404 and 405 are fixed within a baring housing 406, which is connected to a linear drive. In some embodiments, rotating bearings 404 and 405 have a smooth surface. In some embodiments, rotating bearings 404 and 405 may have a gear pattern on the surface that interlocks with a gear pattern on the surface of roller wheel 401. In some embodiments, the roller clamp 402 is held fixed to the control unit 27 (not shown in FIG. 4 for simplicity) via mechanical interlock or surface friction (e.g., rubber pads under sufficient spring preload to firmly grasp the roller body housing and not slip). As the linear drive actuates, the bearing housing 406 is moved back and forth as desired which adjusts the roller wheel's 401 position on the connector tube 403. This change in clamping pressure closes or opens the flow, respectively. Accordingly, the roller clamp control mechanism assembly 400, controlled by control unit 27, effectively limits the flow through the connector tubing 403 to the desired rate. In some embodiments, spring pin 407 attaches the roller clamp 402 to the control unit 27.

The fluid flow rate may be quickly adjusted by way of the control unit 27 to a fully blocked flow rate of 0 ml/hr (e.g., quick occlusion), or any other desired fluid flow rate between 250 ml/hr and 0 ml/hr (e.g., 50 ml/hr, 125 ml/hr), when the roller wheel 301 is moved into the fully engaged position. Thus, the roller wheel 401 may be configured as a substantially binary flow switch (e.g., on/off switch), for example, where the flow rate is either wide open or adjusted down to a specific flow rate such as 250 ml/hr, or even to a fully blocked flow rate of 0 ml/hr.

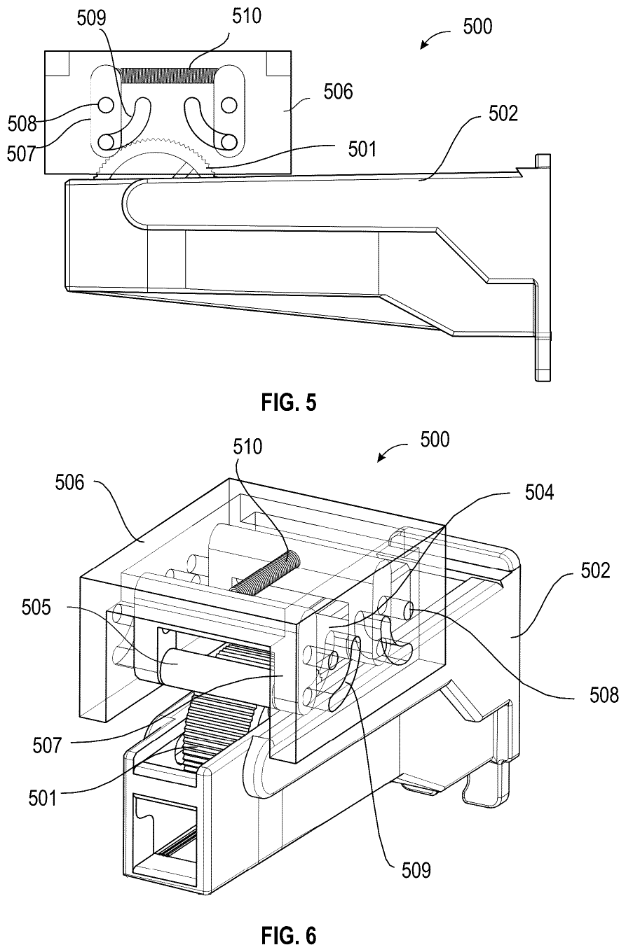

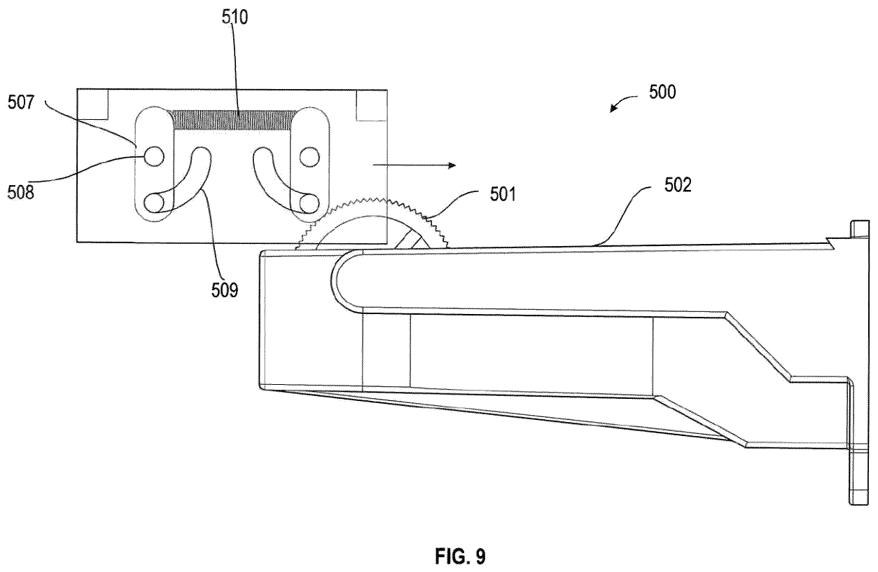

The roller clamp control mechanism assembly 500 illustrated in FIGS. 5-14 may have a body fixture 502 comprising two opposing side walls and a roller clamp assembly 26 may be seated inside the body fixture 502. In some embodiments, the roller clamp assembly 26 comprises roller wheel 501. In some embodiments, two rotating bearings 504 and 505 are fixed within their own respective bearing arm 507, which can pivot about the bearing arm fixed axis 508. In some embodiments, rotating bearings 504 and 505 have a smooth surface. In some embodiments, rotating bearings 504 and 505 may have a gear pattern on the surface that interlocks with a gear pattern on the surface of roller wheel 501. In some embodiments, as the bearing arm 507 rotates, the bearings 504 and 505 are able to slide through the bearing clearance path 509. In some embodiments, the bearing clearance path 509 allows the bearing arm 507 to rotate in one direction (e.g., inwards) but not in the opposite direction (e.g., outwards). In some embodiments, the entire bearing assembly is therefore able to slide over a roller wheel 501, and snap to the roller wheel's 501 position as shown in FIGS. 9-14. As the bearing assembly slides, the bearing housing 506 is moved back and forth as desired which adjusts the roller wheel's 501 position on the connector tube. This change in clamping pressure closes or opens the flow, respectively. Accordingly, the roller clamp control mechanism assembly 500, controlled by control unit 27, effectively limits the flow through the connector tubing to the desired rate. In some embodiments, the bearing arms are attached by a bearing arm return spring 510. The bearing arm return spring 510 biases the bearing arms 507 to return to an upright position.

The fluid flow rate may be quickly adjusted by way of the control unit 27 to a fully blocked flow rate of 0 ml/hr (e.g., quick occlusion), or any other desired fluid flow rate between 250 ml/hr and 0 ml/hr (e.g., 50 ml/hr, 125 ml/hr), when the roller wheel 501 is moved into the fully engaged position. Thus, the roller wheel 501 may be configured as a substantially binary flow switch (e.g., on/off switch), for example, where the flow rate is either wide open or adjusted down to a specific flow rate such as 250 ml/hr, or even to a fully blocked flow rate of 0 ml/hr.

Embodiments of the present disclosure provide a method of adjusting a fluid flow rate through a connector tube coupled to a fluid source. The method may comprise inserting the connector tube through a housing of a roller clamp assembly having a roller wheel and a roller clamp control mechanism which adapts to the roller clamp to control the position of the roller clamp, and controlling the roller clamp control mechanism using a control unit to compress the connector tubing with the roller wheel to produce an adjustment that causes a change in the fluid flow rate through the connector tube.

In some embodiments, the roller clamp control mechanism may comprise a control unit, two rotating bearings fixed to a housing of the control unit, and a body fixture attached to the roller clamp and positioned into the control unit which is connected to a linear drive. The roller clamp may be placed into the housing of the control unit such that the roller wheel is between the two rotating bearings. In some embodiments, as the linear drive actuates, the body fixture is driven forwards or backwards, while the roller wheel stays stationary with respect to the control unit. As a result, the clamping pressure on the connector tube changes.

In some embodiments, the roller clamp control mechanism comprises a control unit and two rotating bearings fixed within a bearing housing. In some embodiments, the bearing hosing is connected to a linear drive, and as the linear drive actuates, the bearing housing is moved back and forth as desired which adjusts the roller wheel's position on the connector tube.

In some embodiments, the roller clamp control mechanism comprises two rotating bearings each fixed within a respective bearing arm which pivots about a fixed axis of the bearing arm and a bearing clearance path for the rotating bearings to slide through. In some embodiments, the bearing clearance path allows the bearing arm of each rotating bearing to rotate inward but not in outward. The roller clamp assembly is then able to slide over the roller wheel and adjust the roller wheel position.

Various examples of aspects of the disclosure are described as numbered clauses (1, 2, 3, etc.) for convenience. These are provided as examples and do not limit the subject technology. Identification of the figures and reference numbers are provided below merely as examples for illustrative purposes, and the clauses are not limited by those identifications.

Clause 1: A roller clamp control mechanism assembly, comprising a housing configured to receive a portion of a connector tube of an infusion set, the housing comprising two opposing side walls spaced apart from each other, each side wall having an opposing guide groove longitudinally positioned in an interior surface, a guide wall disposed between the side walls, the guide wall converging along its length toward the position of the guide grooves, a roller wheel having two axial projections slidingly seated in the guide grooves, the roller configured to move along a longitudinal axis of the housing as the projections slide in the guide grooves, wherein spacing between the guide wall and the roller wheel decreases over a length of the guide wall, and a roller clamp control mechanism which adapts to the roller clamp to control the position of the roller clamp.

Clause 2: The roller clamp assembly of Clause 1, wherein the roller clamp control mechanism comprises a control unit, two rotating bearings fixed to a housing of the control unit, and a body fixture attached to the roller clamp and positioned into the control unit which is connected to a linear drive, wherein the roller clamp is placed into the housing of the control unit such that the roller wheel is between the two rotating bearings, and wherein as the linear drive actuates the body fixture is driven forwards or backwards, while the roller wheel stays stationary with respect to the control unit, thereby changing the clamping pressure on the connector tube.

Clause 3: The roller clamp assembly of Clause 2, wherein the body fixture attaches to the roller clamp via a mechanical interlock or surface friction.

Clause 4: The roller clamp assembly of Clause 2, wherein sliding of the roller wheel in a direction of lesser spacing between the guide wall and the roller wheel causes the roller wheel to impinge on the connector tube to a gradually increasing extent, wherein the increased impingement of the roller wheel on the connector tube is configured to reduce a fluid flow rate through the connector tube.

Clause 5: The roller clamp assembly of Clause 2, wherein the rotating bearings have a gear pattern on the surface that interlocks with a gear pattern on the surface of the roller wheel.

Clause 6: The roller clamp assembly of Clause 2, wherein the roller wheel is configured as a substantially binary flow switch, for example, where the flow rate is either wide open, adjusted down to a specific flow rate, or a fully blocked flow rate.

Clause 7: The roller clamp assembly of Clause 2 further comprising a spring pin that connects the control unit to the linear drive.

Clause 8: The roller clamp assembly of Clause 1, wherein the roller clamp control mechanism comprises a control unit, and two rotating bearings fixed within a bearing housing, wherein the bearing hosing is connected to a linear drive, and wherein as the linear drive actuates, the bearing housing is moved back and forth as desired which adjusts the roller wheel's position on the connector tube.

Clause 9: The roller clamp assembly of claim 8, wherein the rotating bearings attach to the roller clamp via a mechanical interlock or surface friction.

Clause 10: The roller clamp assembly of claim 8, wherein sliding of the roller wheel in a direction of lesser spacing between the guide wall and the roller wheel causes the roller wheel to impinge on the connector tube to a gradually increasing extent, wherein the increased impingement of the roller wheel on the connector tube is configured to reduce a fluid flow rate through the connector tube.

Clause 11: The roller clamp assembly of Clause 8 further comprising a spring pin that attaches the roller clamp to the control unit.

Clause 12: The roller clamp assembly of Clause 1, wherein the roller clamp control

mechanism comprises two rotating bearings each fixed within a respective bearing arm which pivots about a fixed axis of the bearing arm; and a bearing clearance path for the rotating bearings to slide through,

wherein bearing clearance path allows the bearing arm of each rotating bearing to rotate inward but not in outward, and

wherein the roller clamp assembly is able to slide over the roller wheel and adjust the roller wheel position.

Clause 13: The roller clamp assembly of Clause 12, wherein sliding of the roller wheel in a direction of lesser spacing between the guide wall and the roller wheel causes the roller wheel to impinge on the connector tube to a gradually increasing extent, wherein the increased impingement of the roller wheel on the connector tube is configured to reduce a fluid flow rate through the connector tube.

Clause 14: The roller clamp assembly of Clause 12, wherein a means of the roller wheel connection of the roller clamp assembly is agnostic to an initial position of the roller wheel.

Clause 15: The roller clamp assembly of Clause 12 further comprising a bearing arm return spring configured to bias the bearing arms to return to an upright position.

Clause 16: A gravity infusion set comprising a piercing spike, a drop chamber, a connector tube, a fitting, and a roller clamp assembly, the roller clamp assembly comprising a housing configured to receive a portion of a connector tube of an infusion set, the housing comprising two opposing side walls spaced apart from each other, each side wall having an opposing guide groove longitudinally positioned in an interior surface, a guide wall disposed between the side walls, the guide wall converging along its length toward the position of the guide grooves, a roller wheel having two axial projections slidingly seated in the guide grooves, the roller configured to move along a longitudinal axis of the housing as the projections slide in the guide grooves, wherein spacing between the guide wall and the roller wheel decreases over a length of the guide wall, and a roller clamp control mechanism which adapts to the roller clamp to control the position of the roller clamp.

Clause 17: A method of adjusting a fluid flow rate through a connector tube coupled to a fluid source, the method comprising inserting the connector tube through a housing of a roller clamp assembly having a roller wheel and a roller clamp control mechanism which adapts to the roller clamp to control a position of the roller clamp, controlling the roller clamp control mechanism using a control unit to compress the connector tubing with the roller wheel to produce an adjustment that causes a change in the fluid flow rate through the connector tube.

Clause 18: The method according to Clause 17, wherein the roller clamp control mechanism comprises a control unit, two rotating bearings fixed to a housing of the control unit, and a body fixture attached to the roller clamp and positioned into the control unit which is connected to a linear drive, wherein the roller clamp is placed into the housing of the control unit such that the roller wheel is between the two rotating bearings, and wherein as the linear drive actuates the body fixture is driven forwards or backwards, while the roller wheel stays stationary with respect to the control unit, thereby changing the clamping pressure on the connector tube.

Clause 19: The method according to Clause 17, wherein the roller clamp control mechanism comprises a control unit, and two rotating bearings fixed within a bearing housing, wherein the bearing hosing is connected to a linear drive, and wherein as the linear drive actuates, the bearing housing is moved back and forth as desired which adjusts the roller wheel's position on the connector tube.

Clause 20: The method according to Clause 17, wherein the roller clamp control mechanism comprises two rotating bearings each fixed within a respective bearing arm which pivots about a fixed axis of the bearing arm, and a bearing clearance path for the rotating bearings to slide through, wherein bearing clearance path allows the bearing arm of each rotating bearing to rotate inward but not in outward, and wherein the roller clamp assembly is able to slide over the roller wheel and adjust the roller wheel position.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A roller clamp control mechanism assembly, comprising:
a housing configured to receive a portion of a connector tube of an infusion set, the housing comprising:
two opposing side walls spaced apart from each other, each side wall having an opposing guide groove longitudinally positioned in an interior surface;
a guide wall disposed between the side walls, the guide wall converging along its length toward the position of the guide grooves;
a roller wheel having two axial projections slidingly seated in the guide grooves, the roller configured to move along a longitudinal axis of the housing as the projections slide in the guide grooves, wherein spacing between the guide wall and the roller wheel decreases over a length of the guide wall; and
a roller clamp control mechanism which adapts to the roller clamp to control the position of the roller clamp.

2. The roller clamp assembly of claim 1, wherein the roller clamp control mechanism comprises:
a control unit;
two rotating bearings fixed to a housing of the control unit; and
a body fixture attached to the roller clamp and positioned into the control unit which is connected to a linear drive,
wherein the roller clamp is placed into the housing of the control unit such that the roller wheel is between the two rotating bearings, and
wherein as the linear drive actuates the body fixture is driven forwards or backwards, while the roller wheel stays stationary with respect to the control unit, thereby changing a clamping pressure on the connector tube.

3. The roller clamp assembly of claim 2, wherein the body fixture attaches to the roller clamp via a mechanical interlock or surface friction.

4. The roller clamp assembly of claim 2, wherein sliding of the roller wheel in a direction of lesser spacing between the guide wall and the roller wheel causes the roller wheel to impinge on the connector tube to a gradually increasing extent, wherein an increased impingement of the roller wheel on the connector tube is configured to reduce a fluid flow rate through the connector tube.

5. The roller clamp assembly of claim 2, wherein the rotating bearings have a gear pattern on the surface that interlocks with a gear pattern on the surface of the roller wheel.

6. The roller clamp assembly of claim 2, wherein the roller wheel is configured as a substantially binary flow switch, for example, where the flow rate is either wide open, adjusted down to a specific flow rate, or a fully blocked flow rate.

7. The roller clamp assembly of claim 2 further comprising a spring pin that connects the control unit to the linear drive.

8. The roller clamp assembly of claim 1, wherein the roller clamp control mechanism comprises:
a control unit; and
two rotating bearings fixed within a bearing housing,
wherein the bearing hosing is connected to a linear drive, and
wherein as the linear drive actuates, the bearing housing is moved back and forth as desired which adjusts the roller wheel's position on the connector tube.

9. The roller clamp assembly of claim 8, wherein the rotating bearings attach to the roller clamp via a mechanical interlock or surface friction.

10. The roller clamp assembly of claim 8, wherein sliding of the roller wheel in a direction of lesser spacing between the guide wall and the roller wheel causes the roller wheel to impinge on the connector tube to a gradually increasing extent, wherein an increased impingement of the roller wheel on the connector tube is configured to reduce a fluid flow rate through the connector tube.

11. The roller clamp assembly of claim 8 further comprising a spring pin that attaches the roller clamp to the control unit.

12. The roller clamp assembly of claim 1, wherein the roller clamp control mechanism comprises:
two rotating bearings each fixed within a respective bearing arm which pivots about a fixed axis of the bearing arm; and
a bearing clearance path for the rotating bearings to slide through,
wherein bearing clearance path allows the bearing arm of each rotating bearing to rotate inward but not in outward, and
wherein the roller clamp assembly is able to slide over the roller wheel and adjust the roller wheel position.

13. The roller clamp assembly of claim 12, wherein sliding of the roller wheel in a direction of lesser spacing between the guide wall and the roller wheel causes the roller wheel to impinge on the connector tube to a gradually increasing extent, wherein an increased impingement of the roller wheel on the connector tube is configured to reduce a fluid flow rate through the connector tube.

14. The roller clamp assembly of claim 12, wherein a means of a roller wheel connection of the roller clamp assembly is agnostic to an initial position of the roller wheel.

15. The roller clamp assembly of claim 12 further comprising a bearing arm return spring configured to bias the bearing arms to return to an upright position.

16. A gravity infusion set comprising:
a piercing spike;
a drop chamber;
a connector tube;
a fitting; and
a roller clamp assembly, the roller clamp assembly comprising:
a housing configured to receive a portion of a connector tube of an infusion set, the housing comprising:
two opposing side walls spaced apart from each other, each side wall having an opposing guide groove longitudinally positioned in an interior surface;
a guide wall disposed between the side walls, the guide wall converging along its length toward the position of the guide grooves;
a roller wheel having two axial projections slidingly seated in the guide grooves, the roller configured to move along a longitudinal axis of the housing as the projections slide in the guide grooves, wherein spacing between the guide wall and the roller wheel decreases over a length of the guide wall; and
a roller clamp control mechanism which adapts to the roller clamp to control the position of the roller clamp.

17. A method of adjusting a fluid flow rate through a connector tube coupled to a fluid source, the method comprising:
inserting the connector tube through a housing of a roller clamp assembly having a roller wheel and a roller clamp control mechanism which adapts to the roller clamp to control a position of the roller clamp;
controlling the roller clamp control mechanism using a control unit to compress the connector tubing with the roller wheel to produce an adjustment that causes a change in the fluid flow rate through the connector tube.

18. The method according to claim 17, wherein the roller clamp control mechanism comprises:
a control unit;
two rotating bearings fixed to a housing of the control unit; and
a body fixture attached to the roller clamp and positioned into the control unit which is connected to a linear drive,
wherein the roller clamp is placed into the housing of the control unit such that the roller wheel is between the two rotating bearings, and
wherein as the linear drive actuates the body fixture is driven forwards or backwards, while the roller wheel stays stationary with respect to the control unit, thereby changing a clamping pressure on the connector tube.

19. The method according to claim 17, wherein the roller clamp control mechanism comprises:
a control unit; and
two rotating bearings fixed within a bearing housing,
wherein the bearing hosing is connected to a linear drive, and
wherein as the linear drive actuates, the bearing housing is moved back and forth as desired which adjusts the roller wheel's position on the connector tube.

20. The method according to claim 17, wherein the roller clamp control mechanism comprises:
two rotating bearings each fixed within a respective bearing arm which pivots about a fixed axis of the bearing arm; and
a bearing clearance path for the rotating bearings to slide through,
wherein bearing clearance path allows the bearing arm of each rotating bearing to rotate inward but not in outward, and
wherein the roller clamp assembly is able to slide over the roller wheel and adjust the roller wheel position.
